(19) United States
(12) Patent Application Publication
(10) Pub. No.: US 2025/0256016 A1
(43) Pub. Date: Aug. 14, 2025

(54) METHOD, DIALYSIS MACHINE AND DIALYSIS SYSTEM

(71) Applicant: B. Braun Avitum AG, Melsungen (DE)

(72) Inventors: Waldemar Janik, Melsungen (DE); Silvie Krause, Melsungen (DE); Meik Schellhase, Bebra (DE)

(21) Appl. No.: 19/048,492

(22) Filed: Feb. 7, 2025

(30) Foreign Application Priority Data
Feb. 9, 2024 (DE) 10 2024 103 671.0

(51) Int. Cl. A61M 1/16; G16H 20/40; G16H 40/63

(52) U.S. Cl. CPC A61M 1/1603; G16H 20/40; G16H 40/63; A61M 2205/3334; A61M 2205/505; A61M 2205/584; A61M 2209/10

(57) ABSTRACT

A method for operating a dialysis machine includes operating the dialysis machine in different modes. The dialysis machine has a normal operating mode, in which the dialysis machine needs a normal requirement of at least one operating resource supplied to the dialysis machine, and an eco operating mode, in which the dialysis machine needs an eco requirement of the at least one operating resource supplied to the dialysis machine. The eco requirement is reduced relative to the normal requirement. The method also includes operating the dialysis machine as a function of at least one criterion either in the normal operating mode or in the eco operating mode.

### CROSS-REFERENCE TO RELATED APPLICATION

This application claims priority under 35 U.S.C. § 119 to German Application No. 10 2024 103 671.0, filed on Feb. 9, 2024, the content of which is incorporated by reference herein in its entirety.

### FIELD

The present disclosure relates to a method for operating a dialysis machine and to such a dialysis machine. The present disclosure furthermore relates to a dialysis system comprising such a dialysis machine.

### BACKGROUND

For the operation of a conventional dialysis machine, it is typically supplied with at least one operating resource. The amount of operating resource supplied is typically not adapted according to requirements during operation of the dialysis machine.

### SUMMARY

It is an object of the present disclosure to provide a method for operating a dialysis machine and such a dialysis machine and a dialysis system comprising such a dialysis machine which, in particular each have improved properties. In particular, it is an intention to enable particularly resource-efficient operation of the dialysis machine.

A method according to the present disclosure is used to operate a dialysis machine. The dialysis machine has a normal operating mode. In the normal operating mode, the dialysis machine needs a normal requirement of at least one operating resource supplied to the dialysis machine. Expediently, the normal requirement needed is consumed in the normal operating mode. The dialysis machine has an eco operating mode. In the eco operating mode, the dialysis machine needs an eco requirement of the at least one operating resource supplied to the dialysis machine. Expediently, the eco requirement is consumed in the eco operating mode. The eco requirement is reduced relative to the normal requirement. The eco requirement is thus in particular less than the normal requirement. The method comprises a step according to which the dialysis machine is operated either in the normal operating mode or in the eco operating mode as a function of at least one criterion. In this way, the dialysis machine can be operated particularly resource-efficiently as a function of the at least one criterion, in particular according to requirements.

Expediently, the at least one operating resource is electrical energy and—alternatively or in addition—an operating liquid, in particular comprising or consisting of water.

Expediently, the method may be at least partially computer-implemented, in particular stepwise.

Expediently, the eco operating mode may be understood synonymously as a “resource-saving operating mode”, in particular because it makes it possible to economize on the at least one operating resource relative to the normal operating mode. In other words, the eco operating mode can allow particularly resource-efficient operation of the dialysis machine in comparison with the normal operating mode, in particular according to requirements.

In a configuration of the present disclosure, the method comprises a step according to which the at least one criterion is monitored for activation either of the normal operating mode or of the eco operating mode. The method furthermore comprises a step according to which either the normal operating mode or the eco operating mode is activated as a function of the at least one criterion, in order subsequently to operate the dialysis machine as a function of the at least one criterion either in the normal operating mode or in the eco operating mode. In particular, the dialysis machine may thus be operated directly at the start of its operation as a function of the at least one criterion either in its normal operating mode or in its eco operating mode. Expediently, the activation either of the normal operating mode or of the eco operating mode may take place manually and—alternatively or in addition—automatically. It is also conceivable for the activation to be possible either only automatically or only manually or both automatically and manually.

Expediently, a plurality of criteria may be monitored. If a plurality of criteria are monitored, some or all of these criteria may be weighted differently strongly or equally strongly.

In a further configuration of the present disclosure, the method comprises a step according to which the at least one criterion is monitored for switching between the normal operating mode and the eco operating mode. The method furthermore comprises a step according to which switching between the normal operating mode and the eco operating mode takes place as a function of the at least one monitored criterion, so that the dialysis machine is subsequently operated as a function of the at least one criterion either in the normal operating mode or in the eco operating mode. In this way, the requirement of the at least one operating resource may be adapted during the operation of the dialysis machine, which may lead to advantages in respect of the resource efficiency. Expediently, a change between the operating modes results from the switching.

In a further configuration of the present disclosure, the switching between the normal operating mode and the eco operating mode takes place automatically. Alternatively or in addition, the switching between the normal operating mode and the eco operating mode takes place manually. It also conceivable for the switching to be possible either only automatically or only manually or both automatically and manually.

In a further configuration of the present disclosure, a dialysate is passed through the dialysis machine with a dialysate flow rate during the operation of the dialysis machine, in particular both in the normal operating mode and in the eco operating mode. The dialysate flow rate may be from 100 ml/min to 800 ml/min, in particular both in the normal operating mode and in the eco operating mode. The dialysate flow rate in the eco operating mode is in this case less than in the normal operating mode, in particular within the aforementioned range in both cases.

Expediently, the dialysate flow rate in the eco operating mode corresponds to from 1.0 times to 1.6 times a blood flow rate of blood which is dialysed and/or can be dialysed by means of the dialysis machine. In particular, the dialysate flow rate in the eco operating mode corresponds to from 1.0 times to 1.2 times the blood flow rate. This makes it possible to utilize results of studies which have shown that an increase of the dialysate flow does not result in a substantially higher dialysis effectiveness, and conversely a reduction of the dialysate flow does not substantially reduce the dialysis effectiveness, particularly with regard to a dialysate flow rate of about 500 ml/min which is often employed.

In a further configuration of the present disclosure, the dialysis machine comprises an electronic display device, which in particular is touch-sensitive, for visualizing a current operating mode of the dialysis machine. The current operating mode may in particular be the currently activated operating mode of the dialysis machine. In the eco operating mode, a display brightness of the display device is lower relative to the normal operating mode. The display brightness may thus be dimmed in the eco operating mode relative to the normal operating mode. Alternatively or in addition a colour scheme of the display device is adapted in the eco operating mode relative to the normal operating mode. In particular, if the display device comprises an OLED display, the display device may have a chromatic colour scheme in the normal operating mode whereas the display device has an achromatic, in particular black-and-white, colour scheme in the eco operating mode. In particular since the colour black can be represented in OLED displays by pixels which are unlit, and therefore switched off, electrical energy may be saved in this way. Alternatively or in addition, the display device is deactivated, particular fully, in the eco operating mode relative to the normal operating mode. In particular, the current operating mode is temporarily not visualized by means of the deactivated display device.

In a further configuration of the present disclosure, the dialysis machine comprises a sensor device. In particular, the sensor device is configured to detect a quantity for the at least one criterion. Either the quantity for the at least one criterion may directly reflect fulfilment of the at least one criterion, or the fulfilment of the at least one criterion may be identified as a result of a comparison of the quantity with an associated threshold value. The sensor device comprises a brightness sensor, which is configured to detect an ambient brightness of the dialysis machine. The ambient brightness may be a quantity for the at least one criterion. Alternatively or in addition, the at least one criterion may be a predetermined threshold value of the ambient brightness. The sensor device comprises alternatively or in addition a proximity sensor, which is configured to detect a person who is approaching the dialysis machine. A distance of the person from the dialysis machine may in this case be a quantity for the at least one criterion. Alternatively or in addition, the at least one criterion may be a predetermined threshold value of the distance. The sensor device alternatively or in addition comprises a contact sensor, which is configured to detect a tactile, in particular manual, input of a quantity for the at least one criterion. The contact sensor may be incorporated, in particular integrally and/or functionally, by the touch-sensitive display device.

In a further configuration of the present disclosure, the dialysis machine has a flushing mode which can be activated in the normal operating mode and in the eco operating mode, in particular selectively and/or temporarily in each case. The flushing mode is used to flush the dialysis machine with a flushing liquid. The flushing liquid may comprise or be the operating liquid. When the flushing mode is activated during operation of the dialysis machine in the normal operating mode, the flushing liquid is passed through the dialysis machine with a normal flushing temperature, in particular with heat being supplied by means of an electrical heating device of the dialysis machine, and with a flushing liquid flow rate. The normal flushing temperature may be 40° C. The flushing liquid flow rate may be 1 l/min. When the flushing mode is activated during operation of the dialysis machine in the eco operating mode, the flushing liquid is passed through the dialysis machine with an eco flushing temperature which is reduced in relation to the normal flushing temperature, in particular without heat being supplied by means of the electrical heating device, and in particular with the same flushing liquid flow rate. The eco flushing temperature may correspond to a temperature of the flushing liquid which the flushing liquid has when it is taken from a flushing liquid supply system for the dialysis machine. For example, the eco flushing temperature may be 15° C. Reducing the flushing temperature can make it possible to save energy. Expediently, an activation and/or switching of the flushing mode is carried out depending on the at least one criterion and/or in a time-controlled manner.

In a further configuration of the present disclosure, the dialysis machine has a hot-cleaning mode which can be activated in the normal operating mode and in the eco operating mode, in particular selectively and/or temporarily in each case, the hot-cleaning mode being used for hot cleaning of the dialysis machine with a hot-cleaning liquid. The hot-cleaning liquid may comprise or be the operating liquid. When the hot-cleaning mode is activated during operation of the dialysis machine in the normal operating mode, the hot-cleaning liquid is passed through the dialysis machine with a normal hot-cleaning temperature, in particular with heat being supplied by means of the electrical heating device of the dialysis machine, and with a normal hot-cleaning duration. When the hot-cleaning mode is activated during operation of the dialysis machine in the eco operating mode, the hot-cleaning liquid is passed through the dialysis machine with an eco hot-cleaning temperature which is reduced in relation to the normal hot-cleaning temperature, in particular with heat being supplied by means of the electrical heating device, and with an eco hot-cleaning duration which is extended in relation to the normal hot-cleaning duration. Reducing the hot-cleaning temperature can make it possible to save energy. Expediently, an activation and/or switching of the hot-cleaning mode is carried out depending on the at least one criterion and/or in a time-controlled manner.

Expediently, the hot-cleaning duration and the hot-cleaning temperature for the normal operating mode and the eco operating mode may be adapted as a function of a therapy plan, in particular a digital therapy plan, in particular automatically.

Expediently, the adaptation of the hot-cleaning temperature to the eco hot-cleaning temperature or the normal hot-cleaning temperature, respectively, may take place so that the achievement of a predetermined A₀ value is still ensured, in particular despite the adaptation. Since the reduction of the hot-cleaning temperature under the condition of achieving the same A₀ value as before the reduction is accompanied by an extension of the hot-cleaning duration, it is recommendable to perform the reduction of the hot-cleaning temperature when sufficient time is available before the next therapy. This might, for example, be the case when the next therapy is not expected until the following day.

Expediently, it is conceivable for a notification to be given, in particular by means of the display device of the dialysis machine, the notification reflecting the A₀ value which is presently being achieved.

In a further configuration of the present disclosure, the eco operating mode is based relative to the normal operating mode on at least one eco measure. An eco measure may be a reduction of the dialysate flow rate of the dialysate passed through the dialysis machine. Alternatively or in addition, an eco measure, in particular a different eco measure, may be a reduction of the display brightness of the display device of the dialysis machine. Alternatively or in addition, an eco measure, in particular a different eco measure, may be an adaptation the colour scheme of the display device. Alternatively or in addition, an eco measure, in particular a different eco measure, may be a deactivation of the display device. Alternatively or in addition, an eco measure, in particular a different eco measure, may be a reduction of the flushing temperature during the flushing of the dialysis machine. Alternatively or in addition, an eco measure, in particular a different eco measure, may be an extension of the hot-cleaning duration and a corresponding reduction of the hot-cleaning temperature during the hot cleaning of the dialysis machine. Expediently, an activation and/or switching of at least one eco measure, in particular of a mode comprising the at least one eco measure, is carried out depending on the at least one criterion and/or in a time-controlled manner.

In a further configuration of the present disclosure, the method comprises a step according to which the eco operating mode is configured by in particular manual and—alternatively or in addition—automatic selection and—alternatively or in addition—combination of eco measures for the eco operating mode, in particular as a function of a plurality of criteria.

In a further configuration of the present disclosure, the dialysis machine comprises an interface, in particular an electronic interface, for connection to a superordinate control system fed with external data. The method in this case comprises a step according to which the at least one criterion and—alternatively or in addition—a quantity for the at least one criterion is input through the interface. External data may for example be present electricity costs. The superordinate control system may be adapted to process the external data in order to identify whether the at least one criterion is fulfilled. Alternatively or in addition, a control device of the dialysis machine, in particular an internal control device, may be adapted to process the data in order to identify whether the at least one criterion is fulfilled. It is conceivable for the data to be conditioned by means of the control system and supplied in a conditioned form to the control device, in order then to be processed further by the control device. The control device may comprise an electronic microprocessor device.

A dialysis machine according to the present disclosure comprises an electronic control device, in particular the electronic control device, the control device being adapted and—alternatively or in addition—programmed to carry out a method according to the present disclosure as described above. The advantages of the method according to the present disclosure which have been explained above therefore also apply to the dialysis machine according to the present disclosure. It is to be understood that the dialysis machine may comprise further means for carrying out the present disclosure.

A dialysis system according to the present disclosure comprises a dialysis machine according to the present disclosure as described above. In this respect, the advantages of the dialysis machine according to the present disclosure which have been mentioned above also apply to the dialysis system according to the present disclosure having such a dialysis machine. The dialysis system furthermore comprises at least one control system which is superordinate, in particular relative to the dialysis machine, and which is connected to an interface of the dialysis machine, in particular an electronic interface.

Further advantages and features of the present disclosure may be found in the following description of a preferred exemplary embodiment of the present disclosure, which is represented with the aid of the drawings. Reference signs which are the same refer to components which are the same or similar or functionally equivalent.

It is to be understood that the features mentioned above and those yet to be explained below may be used not only in the combination respectively indicated but also in other combinations or individually, without departing from the scope of the present disclosure.

### BRIEF DESCRIPTION OF THE DRAWINGS

FIG. 1 shows a structural diagram of an embodiment of a dialysis system according to the present disclosure with an embodiment of a dialysis machine according to the present disclosure when carrying out a method performed according to the present disclosure;

FIG. 2 schematically shows a display surface of a display device of the dialysis machine according to FIG. 1;

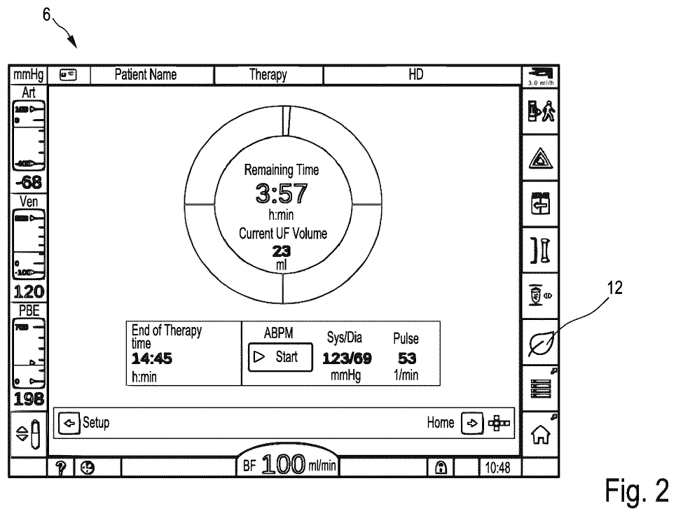

FIG. 3 schematically shows a digital therapy plan for operation of the dialysis machine according to FIG. 1; and

FIG. 4 shows the profile of various A₀ values in a time/temperature diagram.

### DETAILED DESCRIPTION

A dialysis machine 1 has a normal operating mode NM. When the dialysis machine 1 is operated in its normal operating mode NM, the dialysis machine 1 needs a normal requirement NB of at least one operating resource R supplied to the dialysis machine 1.

The dialysis machine 1 furthermore has an eco operating mode EM. When the dialysis machine 1 is operated in its eco operating mode EM, the dialysis machine 1 needs an eco requirement EB of the at least one operating resource R supplied to the dialysis machine 1. The eco requirement EB is reduced relative to the normal requirement NB. Any requirement EB, NB of the at least one operating resource R may correspond to an amount of the operating resource R required for the operation of the dialysis machine 1.

The at least one operating resource R is, for example, electrical energy E. Alternatively or in addition, the at least one operating resource R is an operating liquid L. The operating liquid L may comprise water W or consist of water W.

The dialysis machine 1 comprises an electronic control device 2. The electronic control device 2 is adapted and/or programmed to carry out a method for operating the dialysis machine 1. According to the method, the dialysis machine 1 is operated as a function of at least one criterion K either in the normal operating mode NM or in the eco operating mode EM. A plurality of criteria K may be monitored. If a plurality of criteria K are monitored, they may be weighted differently strongly or equally.

For example, the at least one criterion K is monitored for activation either of the normal operating mode NM or of the eco operating mode EM. Either the normal operating mode NM or the eco operating mode EM is in this case activated as a function of the at least one criterion K. After the activation either of the normal operating mode NM or of the eco operating mode EM, the dialysis machine 1 is operated as a function of the at least one criterion K either in the normal operating mode NM or in the eco operating mode EM. The activation either of the normal operating mode NM or of the eco operating mode EM may take place manually and/or automatically.

For example, the at least one criterion K is monitored for switching between the normal operating mode NM and the eco operating mode EM. Switching between the normal operating mode NM and the eco operating mode EM in this case takes place as a function of the at least one monitored criterion K, in such a way that the dialysis machine 1 is subsequently operated as a function of the at least one criterion K either in the normal operating mode NM or in the eco operating mode EM.

For example, switching between the normal operating mode NM and the eco operating mode EM takes place automatically. Alternatively or in addition, switching between the normal operating mode NM and the eco operating mode EM may take place manually.

For example, while the dialysis machine 1 is being operated, a dialysate D is passed through the dialysis machine 1. The dialysate D passed through the dialysis machine 1 may have a dialysate flow rate, the dialysate flow rate being for example from 100 ml/min to 800 ml/min. The dialysate flow rate in the eco operating mode EM is, for example, less than in the normal operating mode NM.

The dialysate flow rate in the eco operating mode EM may correspond to from 1.0 times to 1.6 times, in particular from 1.0 times to 1.2 times, a blood flow rate of blood B dialysed by means of the dialysis machine 1.

For example, the dialysate D and the dialysed blood B are passed through a dialyser 13 of the dialysis machine 1, in the present case in counterflow.

The dialysis machine 1 comprises for example an electronic display device 6. The display device 6 is in the present case configured to be touch-sensitive. The display device 6 may comprise a touchscreen. The display device 6 is, for example, configured to visualize a current operating mode NM, EM of the dialysis machine 1.

In the eco operating mode EM, a display brightness of the display device 6 may be lower than in the normal operating mode NM. Alternatively or in addition, a colour scheme of the display device 6 in the eco operating mode EM may be modified in relation to the normal operating mode NM. Alternatively or in addition, the display device 6 may be deactivated in the eco operating mode EM whereas the display device 6 is activated in the normal operating mode NM.

For example, the display device 6 comprises an OLED display. The colour scheme of the OLED display may be set to “black-and-white” in the eco operating mode EM. Conversely, the colour scheme of the OLED display may be set to coloured display and/or to “chromatic” in the normal operating mode NM. In the OLED display, the colour black for the black-and-white colour scheme of the eco operating mode EM may be represented by pixels of the OLED display which are which are unlit and/or switched off.

For example, the dialysis machine 1 comprises a sensor device 3. The sensor device 3 may—as in the present case—be configured to detect a quantity G for the at least one criterion K.

In the present case, the sensor device 3 comprises a brightness sensor 7 for detecting an ambient brightness of the dialysis machine 1. The ambient brightness may in this case be a quantity G for the at least one criterion K. Alternatively or in addition, the at least one criterion K may be a predetermined threshold value of the ambient brightness.

The sensor device 3 in the present case comprises a proximity sensor 8 for detecting a person who is approaching the dialysis machine 1. A distance of the person from the dialysis machine 1 may in this case be a quantity G for the at least one criterion K. Alternatively or in addition, the at least one criterion K may be a predetermined threshold value of the distance.

The sensor device 3 in the present case comprises a contact sensor 12 for detecting a tactile, in particular manual, input of a quantity G for the at least one criterion K. The contact sensor 12 may be incorporated by the touch-sensitive display device 6. In the present case, the contact sensor 12 is integrally and functionally incorporated by the touch-sensitive display device 6. For example, the contact sensor 12 may be formed by a button section, in particular of a touchscreen, of the touch-sensitive display device 6.

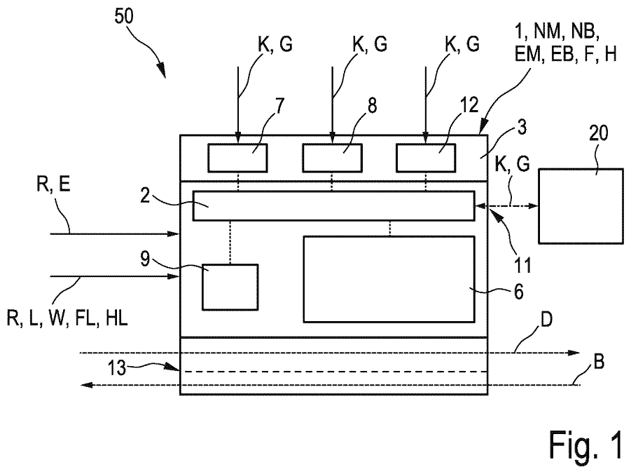

It is to be understood that one or more of the sensors of the sensor device 3 may be omitted from embodiments which differ from that of FIG. 1.

The dialysis machine 1 has, for example, a flushing mode F for flushing of the dialysis machine 1 with flushing liquid FL. The flushing liquid FL may comprise or be operating liquid L. The flushing mode F can be activated in the normal operating mode NM and in the eco operating mode EM, in particular selectively in each case.

When the flushing mode F is activated during the normal operating mode NM, the flushing liquid FL is passed through the dialysis machine 1 with a normal flushing temperature and with a flushing liquid flow rate. In the present case, the dialysis machine 1 comprises an electrical heating device 9, by means of which heat is supplied to the flushing liquid FL when the flushing mode F is activated during the normal operating mode NM. The normal flushing temperature is, for example, 40° C. The flushing liquid flow rate is, for example, 1 l/min.

When the flushing mode F is activated during the eco operating mode EM, the flushing liquid is passed through the dialysis machine 1 with an eco flushing temperature which is reduced in relation to the normal flushing temperature, and with the—in the present case the same—flushing liquid flow rate. In the present case, no heat is supplied to the flushing liquid FL by means of the electrical heating device 9 when the flushing mode F is activated during the eco operating mode EM. The eco flushing temperature may, for example, be 15° C. In particular, the eco flushing temperature corresponds to a flushing liquid intake temperature upstream of the dialysis machine 1.

For example, the dialysis machine 1 has a hot-cleaning mode H for hot cleaning of the dialysis machine 1 with a hot-cleaning liquid HL. The hot-cleaning liquid HL may comprise operating liquid L or be operating liquid L. The hot-cleaning mode H can be activated in the normal operating mode NM and in the eco operating mode EM, for example selectively in each case.

When the hot-cleaning mode H is activated during the normal operating mode NM, the hot-cleaning liquid HL is passed through the dialysis machine 1 with a normal hot-cleaning temperature and with a normal hot-cleaning duration. Heat may in this case be supplied to the hot-cleaning liquid HL by means of the heating device 9 of the dialysis machine 1 in order to heat the hot-cleaning liquid HL to the normal hot-cleaning temperature.

When the hot-cleaning mode H is activated during the eco operating mode EM, the hot-cleaning liquid HL is passed through the dialysis machine 1 with an eco hot-cleaning temperature, which is reduced in relation to the normal hot-cleaning temperature, and with an eco hot-cleaning duration which is extended in relation to the normal hot-cleaning duration. In particular, heat is supplied to the hot-cleaning liquid HL by means of the heating device 9 when the hot-cleaning mode H is activated in the eco operating mode EM, albeit in particular less heat than when the hot-cleaning mode H is activated in the normal operating mode NM.

For example, the hot-cleaning temperature and the hot-cleaning duration for the normal operating mode NM and for the eco operating mode EM may respectively be selected so that an identical A₀ value can be achieved both in the normal operating mode NM and in the eco operating mode EM. In particular, the hot-cleaning duration for achieving a predetermined A₀ value increases exponentially with a decreasing hot-cleaning duration.

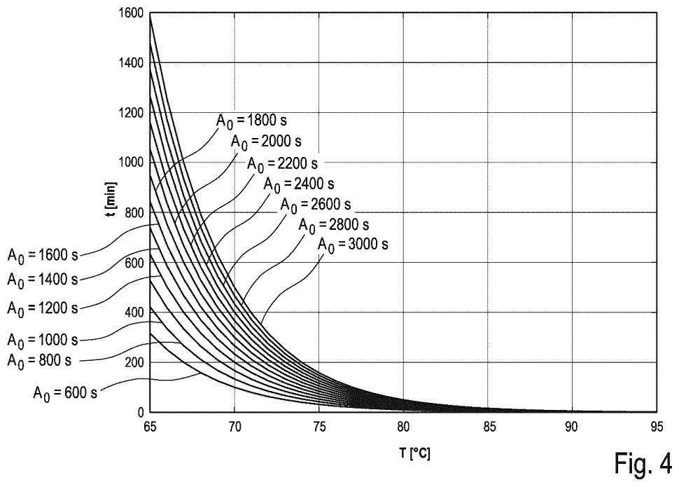

Combinations of hot-cleaning temperatures and hot-cleaning durations for achieving a respective A₀ value may be found by way of example in the diagram shown in FIG. 4. For example, an A₀ value of 600 s may accordingly be achieved by a hot-cleaning temperature of 90° C. with a hot-cleaning duration of 1 min or by a hot-cleaning temperature of 80° C. with a hot-cleaning duration of 10 min or by a hot-cleaning temperature of 70° C. with a hot-cleaning duration of 100 min. If the A₀ value is to be established at 3000 s, the hot-cleaning temperature may for example be 90° C. with a hot-cleaning duration of 5 min or 80° C. with a hot-cleaning duration of 50 min or 70° C. with a hot-cleaning duration of 500 min.

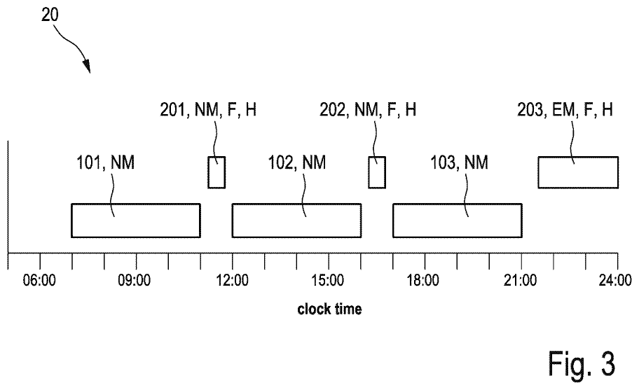

For example, the adaptation of the hot-cleaning duration and of the hot-cleaning temperature may take place as a function of a therapy plan. The therapy plan may be in digital form, as illustrated for example with the aid of FIG. 3. This shows three therapies 101, 102, 103, which are carried out successively on the same day. Between the therapy 101 and the therapy 102, and between the therapy 102 and the therapy 103, there are only time intervals 201, 202 for the normal hot-cleaning duration so that the hot-cleaning mode H is activated in those cases during the normal operating mode NM. Directly after the therapy 103, on the other hand, in the present case there is no further therapy on the same day so that a time interval 203 is available overnight for the eco hot-cleaning duration, for which reason in the present case the hot-cleaning mode H is activated during the eco operating mode EM after the therapy 103.

The eco operating mode EM is based, for example, on at least one eco measure in relation to the normal operating mode NM. An eco measure may in this case be reduction of the dialysate flow rate of the dialysate D passed through the dialysis machine 1. Another eco measure may be reduction of the display brightness of the display device 6 of the dialysis machine 1. Another eco measure may be adaptation the colour scheme of the display device 6. Another eco measure may be deactivation of the display device 6. Another eco measure may be reduction of the flushing temperature during the flushing of the dialysis machine 1. Another eco measure may be extension of the hot-cleaning duration with corresponding reduction of the hot-cleaning temperature during the hot cleaning of the dialysis machine 1.

For example, the eco operating mode EM may be configured by for example manual and/or automatic selection and/or combination of eco measures for the eco operating mode EM. In particular, the eco operating mode EM may be configured as a function of a plurality of criteria K.

The dialysis machine 1 comprises for example an interface 11, in the present case an electronic interface, the interface 11 being configured for connection to a superordinate control system 20 fed with external data. The at least one criterion K and/or a quantity G for the at least one criterion K may in this case be input through the interface 11.

External data may for example be present electricity costs, i.e. costs for the at least one operating resource R in the form of electrical energy. The superordinate control system 20 may in this case be adapted to process the external data in order to identify whether the at least one criterion K is fulfilled. Alternatively or in addition, the control device 2 of the dialysis machine 1, in particular an internal control device, may be adapted to process the data in order to identify whether the at least one criterion K is fulfilled. It is conceivable for the data to be conditioned by means of the control system 20 and supplied in a conditioned form to the control device 2, in order then to be processed further by the control device 2.

The dialysis machine 1 is in the present case part of a dialysis system 50. The dialysis system 50 furthermore comprises the control system 20 superordinate to the dialysis machine 1, the control system 20 being connected to the interface 11 of the dialysis machine 1.

The external data may, for example, be supplied continuously to the superordinate control system 20. The control system 20 may thus be fed continuously with the external data. On the basis of the external data, the control system 20 may decide, in particular automatically, whether an eco measure, and/or which eco measure, should be implemented. For example, a respective eco measure may be activated automatically when a predetermined electricity cost threshold value is exceeded. Alternatively or in addition, predictive analysis models may be used, which can for example predict a future price development on the basis of past developments of the electricity cost.

1. A method for operating a dialysis machine, the dialysis machine having a normal operating mode, in which the dialysis machine needs a normal requirement of at least one operating resource supplied to the dialysis machine, and an eco operating mode, in which the dialysis machine needs an eco requirement of the at least one operating resource supplied to the dialysis machine, which is reduced relative to the normal requirement, the method comprising the step of operating the dialysis machine as a function of at least one criterion either in the normal operating mode or in the eco operating mode.

2. The method according to claim 1, further comprising the steps of:
monitoring the at least one criterion for activation either of the normal operating mode or of the eco operating mode; and
activating either the normal operating mode or the eco operating mode as a function of the at least one criterion, in order to subsequently operate the dialysis machine as a function of the at least one criterion either in the normal operating mode or in the eco operating mode.

3. The method according to claim 1, further comprising the steps of:
monitoring the at least one criterion for switching between the normal operating mode and the eco operating mode; and
switching between the normal operating mode and the eco operating mode as a function of the at least one criterion, so that the dialysis machine is subsequently operated as a function of the at least one criterion either in the normal operating mode or in the eco operating mode.

4. The method according to claim 3, wherein the step of switching between the normal operating mode and the eco operating mode takes place automatically.

5. The method according to claim 3, wherein the step of switching between the normal operating mode and the eco operating mode takes place manually.

6. The method according to claim 1, wherein, during operation of the dialysis machine:
a dialysate is passed through the dialysis machine with a dialysate flow rate, and
the dialysate flow rate in the eco operating mode is less than in the normal operating mode.

7. The method according to claim 6, wherein the dialysate flow rate is in a range of 100 ml/min to 800 ml/min.

8. The method according to claim 6, wherein the dialysate flow rate in the eco operating mode is 1.0 times to 1.6 times a blood flow rate of blood dialysed by the dialysis machine.

9. The method according to claim 1, further comprising the step of displaying a visual parameter on an electronic display device of the dialysis machine to show a current operating mode of the dialysis machine.

10. The method according to claim 9, wherein the visual parameter is a display brightness and/or a color scheme.

11. The method according to claim 9, wherein the electronic display device is deactivated in the eco operating mode.

12. The method according to claim 9, wherein the electronic display device is touch-sensitive.

13. The method according to claim 1, further comprising the step of detecting a quantity for the at least one criterion with a sensor device,
the sensor device comprising:
a brightness sensor for detecting an ambient brightness of the dialysis machine, and/or
a proximity sensor for detecting a person who is approaching the dialysis machine, and/or
a contact sensor for detecting a tactile input of a quantity for the at least one criterion.

14. The method according to claim 1, wherein:
the dialysis machine has a flushing mode for flushing of the dialysis machine with a flushing liquid, the flushing mode being activatable in the normal operating mode and in the eco operating mode,
the flushing liquid is passed through the dialysis machine with a normal flushing temperature and with a flushing liquid flow rate when the flushing mode is activated during the normal operating mode, and
the flushing liquid is passed through the dialysis machine with an eco flushing temperature that is less than the normal flushing temperature and with the flushing liquid flow rate when the flushing mode is activated during the eco operating mode.

15. The method according to claim 1, wherein:
the dialysis machine has a hot-cleaning mode for hot cleaning of the dialysis machine with a hot-cleaning liquid, the hot-cleaning mode being activatable in the normal operating mode and in the eco operating mode,
the hot-cleaning liquid is passed through the dialysis machine with a normal hot-cleaning temperature and with a normal hot-cleaning duration when the hot-cleaning mode is activated during the normal operating mode,
the hot-cleaning liquid is passed through the dialysis machine with an eco hot-cleaning temperature that is less than the normal hot-cleaning temperature, and with an eco hot-cleaning duration that is longer than the normal hot-cleaning duration when the hot-cleaning mode is activated during the eco operating mode.

16. The method according to claim 1, wherein:
the eco operating mode is based relative to the normal operating mode on at least one of the following eco measures:
reducing a dialysate flow rate of a dialysate passed through the dialysis machine,
reducing a display brightness of a display device of the dialysis machine,
adapting a colour scheme of the display device,
deactivating the display device,
reducing a flushing temperature during flushing of the dialysis machine, or
extending a hot-cleaning duration and correspondingly reducing a hot-cleaning temperature during hot cleaning of the dialysis machine.

17. The method according to claim 1, further comprising the step of configuring the eco operating mode by a selection and/or combination of eco measures for the eco operating mode.

18. The method according to claim 1, wherein:
the dialysis machine comprises an interface for connection to a superordinate control system fed with external data; and
the method further comprises the step of inputting the at least one criterion and/or a quantity for the at least one criterion through the interface.

19. A dialysis machine comprising an electronic control device configured to carry out the method according to claim 1.

20. A dialysis system comprising:
a dialysis machine according to claim 19; and
a superordinate control system connected to an interface of the dialysis machine.
